(19) United States
(12) Patent Application Publication
(10) Pub. No.: US 2013/0298903 A1
(43) Pub. Date: Nov. 14, 2013

(54) DISPOSABLE THERMAL THERAPEUTIC APPARATUS AND METHOD OF THERMALLY CONTROLLING THE DELIVERY OF MEDICATION THEREWITH

(71) Applicant: David W. Wright, Denver, CO (US)

(72) Inventor: David W. Wright, Denver, CO (US)

(21) Appl. No.: 13/893,126

(22) Filed: May 13, 2013

Related U.S. Application Data
(60) Provisional application No. 61/646,098, filed on May 11, 2012.

(51) Int. Cl. A61M 5/44; A61M 15/00

(52) U.S. Cl. CPC A61M 5/44; A61M 15/00
USPC 128/200.14; 604/113

(57) ABSTRACT

A portable, disposable, thermal drug dispensing apparatus and method of thermally controlling the delivery of medication therewith is provided. The apparatus provides a predetermined thermal energy over a predetermined amount of time to allow effective drug delivery regardless of the external environment. The apparatus produces exothermic or endothermic thermal energy, in a balanced and controlled environment via a chemical reaction between reactants contained as an integral part within the apparatus. The reactants are provided and automatically combined in a selective, predetermined manner within the apparatus to provide the desired thermal cycle needed to produce the desired, thermal environment for effective drug treatment.

### CROSS-REFERENCE TO RELATED APPLICATION

This application claims the benefit of U.S. Provisional Application Ser. No. 61/646,098 filed May 11, 2012 which is incorporated herein by reference in its entirety.

### BACKGROUND OF THE INVENTION

1. Technical Field

This invention relates generally to medical therapy, and more particularly to apparatus and methods for conducting thermally controlled therapeutic drug delivery.

2. Related Art

Drug therapy is a fundamental component in the field of medicine. Drugs may be administered orally, injected intramuscular, intravenously, topically, transdermal and other methods. Traditional mechanisms operate at ambient temperature, subject to the thermal environment of and about the drug. Mechanisms for delivery of drugs include syringes, infusion bags and pumps for intramuscular and intravenous infusion, nebulizers and delivery tubes for oral or inhalation, patches, and other topical devices for transdermal and cross-membrane delivery and other transport mechanisms. Administered drugs have differing rates of ration based concentrations, temperatures and compound configurations. Prior to and/or during administration, many drugs require cooling to maintain stability or heat to prevent precipitation. In particular, crystallization of chemotherapy drugs during infusion can prevent proper delivery or may result in infusion of unwanted, highly concentrated precipitants.

Thermal control of drugs being delivered is a concern, as made evident by the use of electrically based intravenous drug bag warmers, nebulizer air warmers and other hardware, hardwire-based devices. The process of producing thermal control, be it heating or cooling of a drug or drug delivery modality, be it gas, fluid or solid, requires an elaborate, high cost machine comprised of electrical heating and cooling elements configured in electrical communication with thermal sensors in a closed loop control scheme. These machines are relatively large, immobile and expensive.

Examples:

Heating is used when time-released pharmaceutical drugs such as MSContin (morphine) or OxyContin (oxycodone) are injected to better separate the drug from the waxy filler. Heating may also be used as a means to prevent hypothermia during drug infusion.

Cooling of drugs is required when the environmental temperatures at the point of infusion is elevated, such as in desert combat or in ambulatory settings, for example. Many drugs become ineffective when elevated above body temperature.

### SUMMARY OF THE INVENTION

In accordance with one aspect of the invention, a portable, disposable, thermal drug dispensing apparatus is provided. The apparatus is economical and provides a quick, reliable and economical method of providing temperature control during the process of drug delivery. The apparatus automatically provides a desired predetermined thermal energy over a predetermined amount of time to allow effective drug delivery performance without need of expensive electrical apparatus or other costly modality. The apparatus produces exothermic and/or endothermic thermal energy, in a balanced and controlled environment via a chemical reaction between reactants contained as an integral part within the apparatus. The reactants are provided and automatically combined in a predetermined manner within the apparatus to provide the desired thermal cycle needed to produce the desired, effective thermal environment. Accordingly, the apparatus, in accordance with one aspect of the invention, is wholly self-contained, and thus, is fully functional to provide the desired affect without need of external apparatus.

According to another aspect of the invention, a method of thermally controlling the delivery of medication with a wholly contained apparatus constructed in accordance with the invention without the need of external apparatus is provided. The method includes providing an apparatus containing the desired drug to be delivered and a wholly contained energy source capable of reacting to produce an exothermal and/endothermic thermal energy. Then, causing the energy source to produce one of an exothermal and/or endothermic thermal energy and simultaneously causing the drug to be thermally controlled via the thermal energy. Further, delivering the thermally controlled drug to the intended recipient.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 illustrates a cross-sectional view of a therapeutic drug delivery apparatus constructed in accordance with one aspect of the invention for conducting thermally controlled therapeutic drug delivery;

FIG. 2 illustrates partial cross-sectional view of a therapeutic drug delivery apparatus constructed in accordance with another aspect of the invention for conducting thermally controlled therapeutic drug delivery;

FIG. 3 illustrates an exploded perspective view of a therapeutic drug delivery apparatus constructed in accordance with another aspect of the invention for conducting thermally controlled therapeutic drug delivery;

FIG. 4 is a cross-sectional view of the apparatus of FIG. 3;

FIG. 5 illustrates cross-sectional view of a therapeutic drug delivery apparatus constructed in accordance with another aspect of the invention for conducting thermally controlled therapeutic drug delivery; and

FIG. 6 illustrates cross-sectional view of a therapeutic drug delivery apparatus constructed in accordance with another aspect of the invention for conducting thermally controlled therapeutic drug delivery.

### DETAILED DESCRIPTION OF PRESENTLY PREFERRED EMBODIMENTS

Referring in more detail to the drawings, FIGS. 1-6 illustrate a portable, disposable, thermal drug dispensing apparatus, referred to hereafter as apparatus 10, constructed in accordance with various presently preferred embodiments of the invention, by way of example and without limitation. The apparatus 10 provides a quick, reliable and economical method for delivering a thermally actuated and thermally controlled drug to a patient via energy produced and controlled solely by the apparatus without need of supplemental energy devices or other modalities. The apparatus 10 is both economical in manufacture and in use, is readily portable and further, can be sized to be hand held for single use, whereupon the apparatus 10 is disposable after use, particularly given the low cost associated with its material contents and manufacture.

The apparatus 10 can be provided as an all inclusive device, including an integral exothermic reaction heat producing or endothermic heat reducing and regulating mechanism. The heat production via the exothermic chemical reaction or heat reduction via the endothermic reaction may be achieved by combining two or more elements or chemical substances, known as reactants, contained entirely and integrally within the apparatus 10, which in turn, produce a product and a release of energy or a reduction of energy from the surrounding environment. The change in enthalpy, (thermodynamic potential) for an exothermic reaction is less than zero (<0), and thus, a larger value of energy released in the reaction is subtracted from a smaller value of energy used to initiate the reaction, the opposite being true for an endothermic reaction.

The exothermic reactants may be provided individually as, or as a combination of, solids, liquids and gasses. Some examples include:

Combining anhydrous copper (II) sulfate with water (Solid+Liquid):

CuSO₄+5H₂O→CuSO₄.5H₂O+HEAT; or

Combining oxygen with iron (Gas+Liquid):

4Fe+3O₂→2Fe₂O₃+HEAT.

The endothermic reactants may be provided individually as, or as a combination of, solids, liquids and gasses. An example includes:

Combining citric acid and sodium bicarbonate:

H₃C₆H₅O₇(aq)+3NaHCO₃(s)→3CO₂(g)+3H₂O(l)+Na₃C₆H₅O₇(aq).

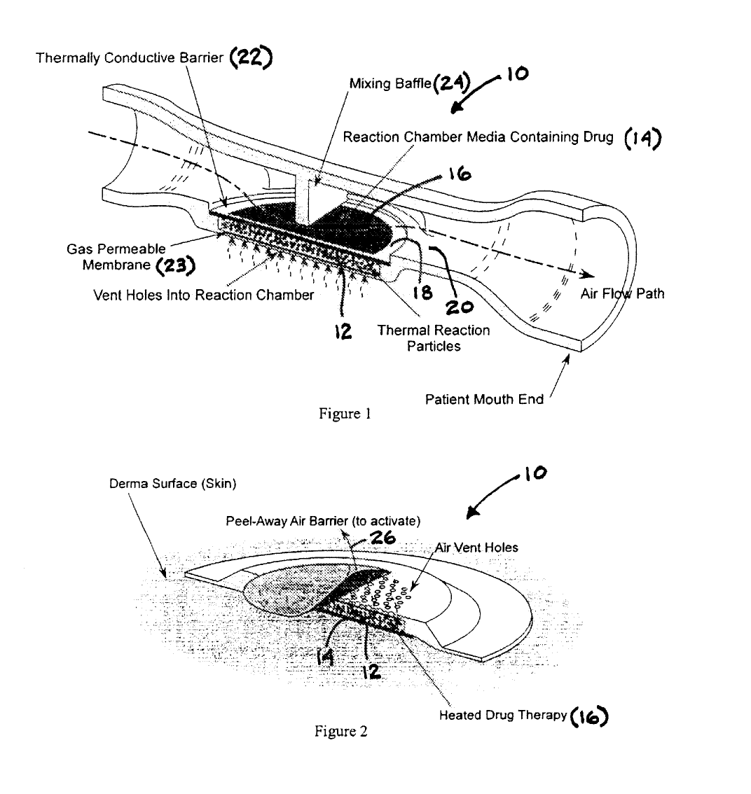

FIG. 1 illustrates an example of an apparatus 10, shown as a nebulizer, containing a thermal control chamber 12 in thermal communication with a drug chamber 14. A drug 16 contained in the drug chamber 14 may be bound within a porous media or applied to the surface 18 of the inner lumen 20 of the nebulizer 10. The thermal energy provided by the reaction may cool and heat the drug 16 or substance being delivered in accordance to design. The thermal energy created by the combined reaction of the first and second reactants may be distributed throughout the inner lumen 20 via conduction of the thermal energy through a thermally conductive barrier layer of material 22 that is disposed between and separates the thermal control chamber 12 from the drug chamber 14 and drug 16. The nebulizer 10 produces the chemical reaction by combining first and second reactants respectively including a solid material and ambient air, however, the reaction may be produced by combining a liquid with a solid or by the combination of two liquids. In the example shown, the reaction is initiated by removal of a gas barrier or membrane (see FIG. 2 for example) acting to initially separate the first and second reactants from one another to prevent the reaction until the desired time of drug treatment. At the desired time, the membrane is selectively removed, thus exposing the first reactive solid to the second reactive air via a gas permeable membrane 23 disposed between the first and second reactants. A baffle 24 is shown to facilitate exposure of the inhaled air across the thermally activated drug 16.

FIG. 2 illustrates an example of an apparatus 10, shown as a transdermal patch, containing at thermal control chamber 12 in thermal communication with a drug chamber 14. The drug 16 may be bound within a porous media or applied to the surface opposite the patient. The thermal energy provided by the reaction may cool and heat the drug 16 or substance being delivered in accordance to design. The thermal reaction can be produced by the combining a solid material in ambient air, as discussed above, however, the reaction may be produced by combining a liquid with a solid or by the combination of two liquids. In the example shown, the reaction is initiated by removal of a gas barrier membrane 26, thus exposing the reactive solid to air.

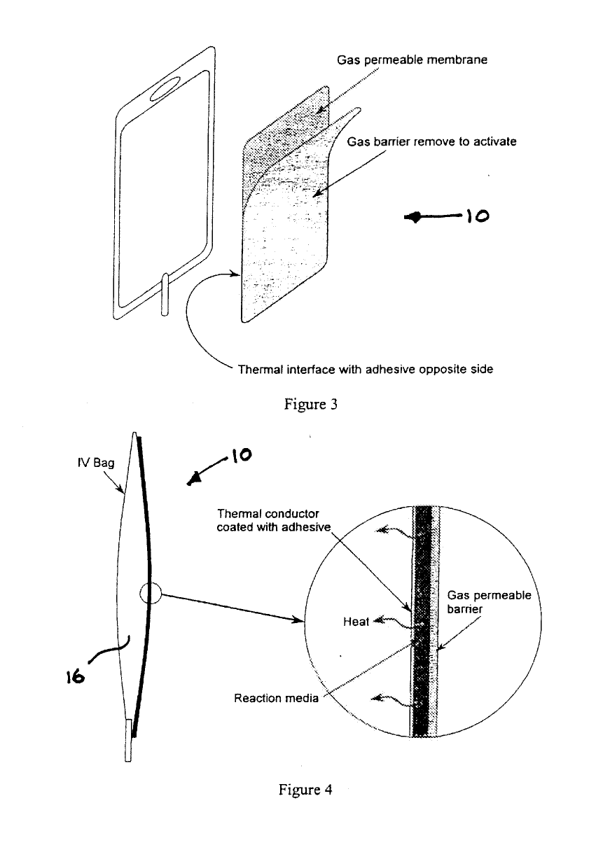

FIG. 3 illustrates an example of an apparatus 10, shown as intravenous infusion bag, with a thermal reaction pad (labeled in FIG. 4 as a thermal conductor coated with adhesive) adhered to an outer surface of the IV bag. The thermal reaction pad is flexible, thus, complies with the flexible contour of the IV bag. It should be recognized that the IV bag can be produced with the thermal reaction pad adhered thereto, or, the thermal reaction pad can be adhered to the IV bag when desired. Upon activating the thermal reaction pad by bringing the first and second reactants into contact with one another, such as discussed above, the thermal affect produced by the thermal reaction pad imparts thermal energy to the drug 16 contained within the IV bag via the thermally conductive barrier that is adhered or otherwise attached to the outer surface of the IV bag.

FIG. 4 illustrates a cross section of the thermal reaction pad as configured with an air activated solid material. In a different embodiment, the reaction chamber containing the solid may be exposed to a liquid, contained within the reaction pad, thus producing the desired thermal energy state.

FIG. 5 illustrates an example of another apparatus 10, shown as a nebulizer that produces a thermal reaction by combining a first reactant solid material with a second reactant liquid 30. In the example shown, the reaction is initiated by depressing a blister 28 and rupturing a membrane 31 underlying the blister 28, wherein the membrane 31 acts to initially separate the reactants from one another, thereby preventing the thermal reaction until the desired, selected time. When desired, the membrane 31 is ruptured by depressing the blister 28 to allow the liquid reactive fluid 30 to pass through an opening 33 to enter a reaction chamber 32 containing a reactive solid to mix with the reactive solid. A thermally conductive barrier 22 isolates and separates the byproducts of the reaction contained in the reaction chamber 32 from the drug 16, while conductively transferring thermal energy to the drug 16 to bring the temperature of the drug 16 to its proper administering temperature. A baffle 24 is shown to facilitate exposure of the inhaled air across the thermally activated drug 16.

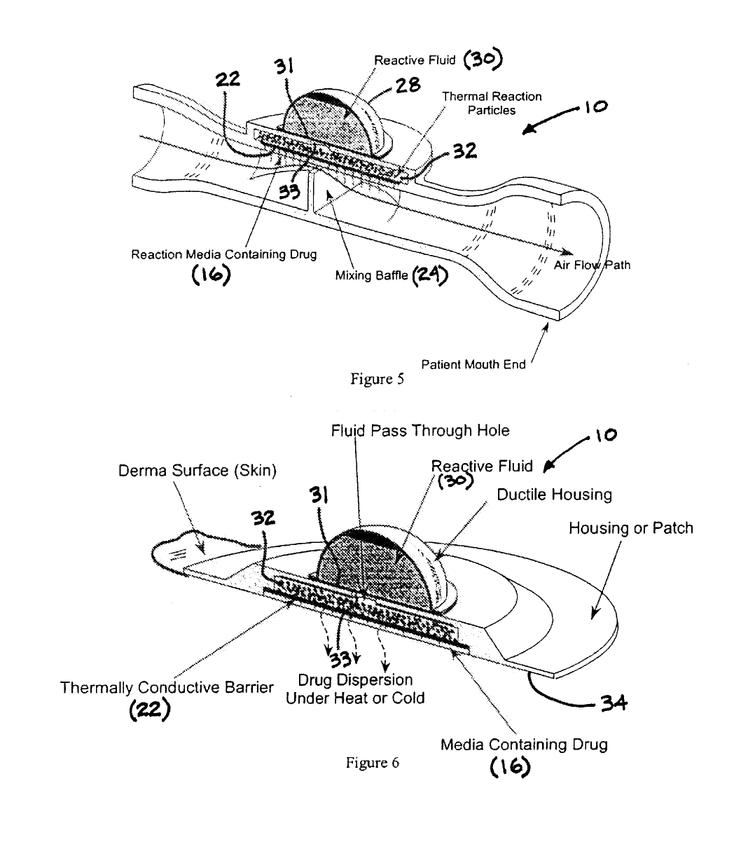

FIG. 6 illustrates an example of another apparatus 10, shown as a transdermal patch that produces a thermal reaction by combining a first reactant solid material with a second reactant liquid. In the example shown, the reaction is initiated by rupturing a membrane 31 underlying a flexible blister 28 by depressing the blister 28, as discussed above, and introducing the reactive fluid 30 into a thermal control chamber 32 containing the reactive solid. A conductive barrier 22 separates the thermal control chamber 32 from an underlying drug 16, such that upon activating the thermal process, thermal energy is conducted through the conductive barrier 22 to the drug 16 to bring the drug 16 to its effective administering temperature. The device 10 shown can be applied to the user's skin surface via an adhesive 34 on an underside of the flexible transdermal drug delivery patch 10, such that the drug therapy is administered to deliver medication across the patient's skin.

Many modifications and variations of the present invention are possible in light of the above teachings. For example, it is contemplated that the thermal control mechanisms discussed and illustrated can be readily applied to other medication delivery apparatus, such as syringes. It is, therefore, to be understood that the invention may be practiced otherwise than as specifically described, and that the scope of the invention is defined by any ultimately allowed claims.

What is claimed is:

1. A portable, disposable, thermal drug dispensing apparatus, comprising:
a thermal control chamber containing a first reactant;
a second reactant;
a membrane disposed between said first reactant and said second reactant;
a drug; and
a conductive barrier disposed between said thermal control chamber and said drug.

2. The apparatus of claim 1 wherein said membrane is gas permeable.

3. The apparatus of claim 1 wherein said second reactant is ambient air.

4. The apparatus of claim 1 wherein said second reactant is liquid.

5. The apparatus of claim 4 wherein said liquid is contained in a flexible blister and said membrane is selectively rupturable upon depressing said flexible blister to bring said first and second reactants into contact with one another.

6. The apparatus of claim 1 wherein said apparatus is a nebulizer.

7. The apparatus of claim 1 wherein said apparatus is a transdermal patch.

8. The apparatus of claim 1 wherein said apparatus is an intravenous bag.

9. The apparatus of claim 8 wherein said conductive barrier is adhered to an outer surface of said intravenous bag.

10. A method of thermally controlling the delivery of a drug, comprising:
providing an apparatus having a thermal control chamber containing a first reactant with a membrane separating the first reactant from a second reactant and further having a conductive barrier separating the thermal control chamber from a drug; and
selectively bringing the first and second reactants into contact with one another to produce a thermal reaction and causing the thermal reaction to bring the drug to its effective administering temperature via conduction of thermal energy through the conductive barrier.

11. The method of claim 10 further including removing the membrane to bring the first and second reactants into contact with one another.

12. The method of claim 11 further including supplying the second reactant as ambient air.

13. The method of claim 10 further including providing the membrane as a gas permeable membrane.

14. The method of claim 10 further including containing the second reactant in a flexible blister and selectively rupturing the membrane by depressing the flexible blister to bring the first and second reactants into contact with one another.

15. The method of claim 14 further including providing the second reactant as a liquid.

16. The method of claim 10 further including providing the apparatus as a hand held nebulizer.

17. The method of claim 10 further including providing the apparatus as a transdermal patch.

18. The method of claim 10 further including providing the apparatus as an intravenous bag.
